(19) United States
(12) Patent Application Publication
(10) Pub. No.: US 2024/0139111 A1
(43) Pub. Date: May 2, 2024

(54) METHODS FOR THE EXTRACTION OF DISPERSIBLE MICROCAPSULES

(71) Applicant: SPOROMEX LIMETED, Bridlington East Riding of Yorkshire (GB)

(72) Inventors: Grahame MacKenzie, Bridlington East Riding of Yorkshire (GB); Terence Simon Corbitt, Bridlington East Riding of Yorkshire (GB); Guy Monroe Entract, Thessaloniki (GR)

(73) Assignee: SPOROMEX LIMITED, Bridlington East Riding of Yorkshire (GB)

(21) Appl. No.: 18/278,890

(22) PCT Filed: Feb. 25, 2022
(86) PCT No.: PCT/GB2022/050515

§ 371 Date: Aug. 25, 2023

(30) Foreign Application Priority Data
Feb. 26, 2021 (GB) 2102754.5
Apr. 29, 2021 (GB) 2106179.1

(51) Int. Cl. A61K 9/50; A61K 9/51; A61K 45/06

(52) U.S. Cl. CPC A61K 9/5063; A61K 9/5192; A61K 45/06

(57) ABSTRACT

A one-pot procedure to extract a dispersible exine shell. The shell can be used as a protection and/or delivery and/or removal vehicle for active substances or as an antioxidant. The invention provides a formulation containing the dispersible exine shell together with an active substance; and a method for preparing the shell by isolating a dispersible exine shell from a naturally occurring spore or pollen grain by treating the spore or pollen grain with a base or surfactant or both with or without a catalyst in the same reaction vessel.

### FIELD OF THE INVENTION

This invention relates to one-pot methods to extract dispersible exine shells, which can be coloured and usable as protection and/or delivery vehicles for active substances where such active substance is not an immunocontraceptive vaccine.

### BACKGROUND TO THE INVENTION

It is known from WO-2005/000280 to use the exine coatings of naturally derived (typically plant) spores or pollen grains as delivery vehicles for active substances such as pharmaceuticals and dietetic substances. These coatings can be isolated from spores or pollen grains by successive treatments, for example with organic solvents, alkalis, and acid under reflux conditions so as to remove the lipid, carbohydrate, protein and nucleic acid components that may be attached to or contained within the exine shell. Enzymatic methods have also been used to isolate the exine coating from other components of a spore or pollen grain.

Exine coatings (or shells) take the form of essentially hollow microcapsules that can be impregnated or filled with, or chemically or physically bound to, another substance. According to WO-2005/000280, a pharmaceutical or dietetic active substance may be physically or chemically bound to, adsorbed on, or more typically encapsulated within such a hollow exine shell. The exine/active substance combination may then be formulated—often mixed with conventional excipients, diluents, or carriers and/or with release rate modifiers—for the desired mode of delivery: for example, oral, buccal or pulmonary delivery.

WO-2007/012857 discloses the use of exine shells as delivery vehicles in topical formulations. This document describes how the exine shells, despite their mechanical and chemical strength, can be caused, by gentle rubbing, to release a substance encapsulated within them. This elastic property makes the exine shells particularly suitable for topical delivery of substances, such as cosmetics or sunscreens, to surfaces such as the skin.

Sometimes, when formulating an active substance, it is necessary to protect the substance, at least temporarily, from external influences such as gastric acid, heat light, moisture, or oxygen (air). This may be for the purpose of improving the storage stability of the formulation, or it may be to ensure that the formulation reaches, following its delivery to a patient, the appropriate part of the body.

Exine shells can themselves provide a degree of protection for an encapsulated active substance; for instance, from atmospheric effects such as light and/or oxygen (air), and therefore from premature degradation. The physical protection they provide can also help reduce loss of the active substance by evaporation, diffusion or leaching. It has also been found (as disclosed in WO-2007/012856) that in many cases an exine shell can itself act as an antioxidant, rather than merely as a physical barrier to oxygen (air), this effect being observable even when an active substance is outside of, rather than encapsulated within, the shell.

In WO-2007/012856 such exine shells isolated by sequential hydrolysis steps of solvent and then base catalysed by such as potassium hydroxide followed by acid hydrolysis (for example, with phosphoric acid) are designated “AHS”. Also disclosed are exine shells designated “BHS,” which were subjected only to solvent and base hydrolysis (for example, with potassium hydroxide). Both AHS and BHS exine shells were extracted under reflux conditions. The BHS samples comprised not only the exine shell but also a proportion of the cellulosic intine layer normally removed during the acid treatment. It was found that some BHS exine coatings were particularly effective in reducing the oxidation rates of oils encapsulated therein when exposed to UV light and air.

Exine shells (coatings) isolated by the hydrolysis processes described in WO-2007/012856 (which did not involve acetolysis) have been found to be more susceptible to oxidative breakdown than those isolated under acetolysis processing conditions such as those used by Erdtman (see G. Erdtman, Svensk Botanisk Tidskrift, 1960, 54, 561-564).

Exine shells isolated using published methods do not quickly disperse when placed in solution. Surprisingly, however, the present inventors have found that dispersible exine shells can be prepared under hydrolysis treatment conditions to an appropriate degree using a one-pot method under aqueous nonacidic (pH>7) nonreflux conditions (<85° C.) without undue structural degradation. Such shells have also been found to retain antioxidant activity. Also, surprisingly, the present inventors have found differences between properties of the exine shell products extracted by the two pot reflux processes (using either organic or aqueous reagents, when used individually or sequentially) as taught in WO-2005/000280 and WO-2007/012856, and those of the products extracted from the one-pot nonreflux 85° C.) and nonacidic processes (pH>7) of the present invention. Of particular note, is the rapid dispersibility in polar solutions of the products from the one-pot processes.

The resultant dispersible exine shells can be suitable for attaching or encapsulating active substances, particularly lipophiles, for their protection and/or delivery, especially in agrochemicals, cosmetics, foods, confectionery and chewing gum, soaps and detergents and pharmaceuticals and veterinary.

### STATEMENTS OF THE INVENTION

According to a first aspect of the invention there is provided a formulation comprising one or more active substances together with a dispersible exine shell of a naturally occurring spore or pollen grain; and wherein the dispersible exine shell is dispersible in solution wherein the active substance is not an immunocontraceptive.

The formulation may in particular be a product of the type: agrochemical; beverage; food product; confectionery or chewing gum product; cosmetic; a household product, soap; detergent; laundry; personal care; toiletry, a diagnostic, pharmaceutical, vaccine or veterinary product; dietetic (which includes nutraceutical).

According to this aspect of the invention the formulation may be obtainable by a process comprising treating a spore or pollen grain with an aqueous non acidic treatment. Preferably, the aqueous non acidic treatment is at a pH greater than pH 7. Preferably, the dispersible exine shell is obtainable without use of an organic solvent.

According to a second aspect of the present invention there is provided a method of preparing a dispersible exine shell, the method involving isolating an exine shell from a naturally occurring spore or pollen grain by treating the spore or pollen grain with an aqueous non acidic treatment with or without a catalyst.

According to this aspect of the invention the method for extracting a dispersible exine shell of naturally occurring spores and pollen grains, is a one-pot process. The method comprising the steps of:
(a) treating “naturally occurring” spores or pollen grains, with an aqueous nonacidic treatment under nonreflux conditions (<85° C.). Preferably the aqueous nonacidic treatment is at a pH greater than pH 7;
(b) in the same reaction vessel (one-pot) where one or more of the components of the mixture can be added at different times in the same reaction vessel;
(c) the dispersible exine shell is extracted without use of an organic solvent;
(d) separating the dispersible exine shells from the mixture after treatment.

The term ‘one-pot’, means that all reagents can be placed into the one reaction vessel either as one mixture or where the ingredients are added at different times during the process.

The method may comprise the treatment being carried out at different temperatures in the same reaction vessel but always less than <85° C. For example, the treatment may be carried out at a temperature of from about 5° C. to less than 85° C. The use of lower temperatures will require a longer period of extraction.

The dispersible exine shell may be chemically modified to alter its properties by such as changing the:
i. surface polarity (e.g. relative availability of protonated or salt forms of surface acidic functional groups and lipophilic groups such as fatty acid chains) to alter its dispersibility in solvents or alter the retention/release characteristics of substances encapsulated within the dispersible exine shell;
ii. surface functional groups to target it to an intended site of administration (for example, to render it more surface-active or more surface-adhesive), or to facilitate its attachment to an active substance. Suitable such chemical modifications may include the attachment of functional groups such as cationic and/or anionic groups and/or functional groups, which increase the affinity of the dispersible exine shell for a surface to which it is intended to be applied and targeted to.

Suitable ways in which a substance may be chemically bound to a dispersible exine shell may involve chemical derivatisation of the dispersible exine shell so as to facilitate its chemical binding to the substance in question. Chemical binding may encompass covalent or other forms of chemical bond, for example hydrogen bonds, sulfide linkages, ionic bonds, van der Waals bonds or dative bonds. Physical binding of an active substance to a dispersible exine shell may include, for example, adsorption (e.g., involving hydrophobic/hydrophilic interactions) of the substance onto a surface (whether internal or external) of the dispersible exine shell.

The formulation may comprise a bioconjugate, that is, a macromolecular complex obtained by attachment, e.g., ionic, hydrogen bonding, hydrophilic/hydrophobic interactions, van de Waals or covalently bonding active substances to a carrier or substrate comprising a dispersible exine shell. Thus, the formulation may include one or more active substances that are chemically or physically bound to the dispersible exine shell. The active substance may be a pharmacologically active substance, i.e. a drug, or may be active in other environments, e.g. a pesticide, and the like. For ease of formation such as ionic bonding, hydrogen bonding, hydrophilic/hydrophobic interactions van der Waals forces or encapsulation within the dispersible exine shells are preferred although in many cases covalent bonding of the active substance to the carrier may be required. The active substance may be reacted directly with the dispersible exine shells or physically attached to produce a bioconjugate. However, in embodiments of this invention the dispersible exine shells may be functionalised so that the active substance can be attached by a suitably stable covalent linkage or other chemical linkage. For example, for oral delivery the linkage may be selected to be stable in acid solutions so that the active substance and support can pass through the stomach into the intestinal tract. Alternatively, active substances that are encapsulated may be stabilised due to protection provided by the dispersible exine shells. Added protection of physically attached or chemically attached active substances may be achieved by an additional coating such as with gum Arabic starch or Eudragit. Conventional film coatings may be used, for example, hydroxypropyl cellulose, shellac or other modified celluloses.

In an embodiment of the invention, the one-pot prepared dispersible exine shell may be intact or substantially so. In other words, apart from the micro- or nano-pores, which are naturally present and penetrate such shells, it will provide a continuous morphology and topography of the outer wall defining an inner cavity into or onto which an active substance can be loaded. However, the dispersible exine shell may be broken or damaged in parts; the invention thus embraces a fragment of a dispersible exine shell. Such broken or damaged fragments of a dispersible exine shell may also be useful in all aspects of the present invention. Therefore, for the avoidance of doubt, reference herein to a dispersible exine shell should be construed as encompassing whole exine shells, broken or damaged fragments of exine shells, and combinations thereof. The dispersible exine shell, whether whole or broken or damaged, is continuous over at least 0.1% or at least 1% or at least 10% or at least 30%, suitably at least 50 or 75 or 80 or 90%, of the surface area, which a dispersible exine shell from the relevant spore or pollen grain species would have if intact.

In an embodiment of the first aspect, the dispersible exine shell is extractable by a one-pot process comprising treating pollen grains or spores with an aqueous nonacidic treatment under nonreflux conditions (<85° C.). Preferably the aqueous nonacidic treatment is at a pH greater than pH 7.

The dispersible exine shell is extracted without use of an organic solvent.

In another aspect of the invention there is provided a dispersible exine shell isolated from a naturally occurring spore or pollen grain wherein the exine shell is dispersible in solution.

The dispersible exine may be derivatised wherein the derivatisation comprises, hydrolysis, salt formation, protonation, deuteration, tritiation, esterification, amination, quarternisation, acetylation, sulfonation, sulfation, thiolation, alkylation, azidation, phosphorylation, nitration, metal chelation, halogenation, hydrogenation or chloromethylation or thiolation or any combination thereof.

A third aspect provides the use of such a dispersible exine shell as a protection and/or delivery vehicle for an active substance. There is also provided the use of a dispersible exine shell as a delivery vehicle for one or more active substances. There is also provided the use of a dispersible exine shell as a removal vehicle for one or more active substances. Thus, there is provided the use of such a dispersible exine shell as a protection and/or delivery and/or removal vehicle for an active substance. One or more active substances may be chemically or physically bound to, or encapsulated within, the cavity of the dispersible exine shell, or the wall of the dispersible exine shell or a combination of these. In an embodiment, it is encapsulated within the dispersible exine shell.

According to a fourth aspect of the present invention the dispersible exine shells, i.e. the empty exine shells, or a fragment thereof can be used for removal, e.g. remediation or sequestration:
(i) to load an active (e.g. an oil from an oil-in-water emulsion or water-in-oil emulsion);
(ii) to remove an active substance, e.g. from solutions and emulsions;
(iii) in the purification of substances from such as fermentation broths or for the remediation of metals;
(iv) to remove, e.g. by removal, pollutants and contaminants (e.g. oils, drugs and agrochemicals);
(v) to remove, e.g. by removal, animal or human bodily fluids and secretions (including such as blood, faeces, saliva, sweat, tears, or urine).

The term “removal” shall include, but shall not be limited to, remediation, sequestration, and the like.

A fifth aspect of the invention provides a dispersible exine shell according to the first aspect, for use in a method of surgery, therapy, prevention or diagnosis practised on a living plant, human or animal body. The dispersible exine shell may thus be used as a protection and/or delivery vehicle for a substance, which is active as an agrochemical, pharmaceutical, veterinary or diagnostic agent.

The invention further provides the use of a dispersible exine shell as an antioxidant for an active substance.

A sixth aspect of the invention provides the use of a dispersible exine shell according to the first aspect, in the manufacture of a medicament for the protection and/or delivery of an agrochemical, pharmaceutically or veterinary active substance or a diagnostic agent to a human or animal patient.

The invention further provides the use of a dispersible exine shell as herein described wherein a protective additive is also together with the dispersible exine shell and the active substance.

The invention further provides the use of a dispersible exine shell as herein described wherein a protective additive is, together with the active substance, chemically or physically bound to the dispersible exine shell.

The invention further provides the use of a dispersible exine shell as herein described wherein a protective additive is, together with the active substance, encapsulated within the dispersible exine shell or within the shell wall.

The invention further provides the use of a dispersible exine shell as herein described wherein the outside of the dispersible exine shell is further coated with a material to aid retention of the active substance.

The invention further provides the use of a dispersible exine shell as herein described, or a fragment thereof, in the sequestration of an active substance.

The invention further provides the use of a dispersible exine shell as herein described, or a fragment thereof, in the sequestration of an active substance from solutions and emulsions.

The invention further provides the use of a dispersible exine shell as herein described, or a fragment thereof, in the remediation of pollutants and contaminants.

The invention further provides the use of a dispersible exine shell as herein described, or a fragment thereof, to remove animal or human bodily fluids and secretions (including such as blood, faeces, saliva, sweat, tears, or urine).

The invention further provides the use of a dispersible exine shell as herein described in the manufacture of a formulation for the protection and/or delivery of an active substance to a living organism.

The invention further provides the use of a dispersible exine shell as herein described in the manufacture of a formulation for the protection and/or delivery of an active substance to a non-living material.

The invention further provides the use of a dispersible exine shell as herein described for use in a method of surgery, therapy, prevention or diagnosis practised on a living plant, human or animal body.

According to a seventh aspect of the present invention there is provided a method for protecting an active substance from oxidation, light and/or for increasing the stability of the active substance or of a composition containing it, the method comprising formulating the active substance with a dispersible exine shell according to the first aspect of the invention.

The invention further provides a method of increasing the oxidative stability of an active substance, which comprises adding a dispersible exine shell as herein described to the active substance.

The present inventors have found surprisingly that dispersible exine shells can be prepared under hydrolysis reaction conditions by treating a pollen grain or spore with either an alkali or a surfactant or both, with or without a catalyst, in the same reaction vessel where one or more of the components of the mixture can be added at the same time or different times in the same reaction vessel. Surprisingly, some of the properties of the dispersible exine shells had some distinguishable differences. For example, the one-pot dispersible exine shell product dispersed in aqueous solution whereas the two-pot exine shell product did not under the same conditions. Also, the one-pot dispersible exine shell product dispersed particularly well in a polar solvent when a highly lipophilic substance is encapsulated within the dispersible exine shell.

There is particularly provided a dispersible exine shell of a naturally occurring spore or pollen grain wherein the dispersible exine shells exhibit a dispersion time of up to 60 seconds. This contrasts to little or no dispersibility found with exine shells prepared under reflux and/or multi-pot conditions. The data in Table 1 herein illustrate that that exine shells of a naturally occurring spore or pollen grain prepared under reflux conditions, e.g. empty exine shells, not loaded with an active substance, disperse faster than exine shells prepared under reflux and/or multi-pot conditions. The extraction time may be from 10 minutes to 24 hours; or 1 hour to 24 hours; or 2 hours to 24 hours; or 4 hours to 24 hours; or 8 hours to 24 hours; or 12 hours to 24 hours; or 16 hours to 24 hours.

An exine shell is the outer coating from around a naturally occurring (“raw”) spore or pollen grain. It may consist in part or mainly of sporopollenin or a derivative of it. It may be of a type described in WO-2005/000280.

According to the present invention, the dispersible exine shell may be derived from any suitable naturally occurring spore or pollen grain. In this context, the term “plant” is to be construed in its broadest sense, and embraces for example mosses, fungi, algae, gymnosperms, angiosperms and pteridosperms. Moreover, the term “spore” is used to encompass not only true spores such as are produced by ferns, mosses and fungi, but also pollen grains, as are produced by seed-bearing plants (spermatophytes) and endospores of organisms such as bacteria. Similarly, the term “naturally occurring” means that a spore is produced by a living organism, whether prokaryote or eukaryote and whether plant or animal. The spore (which term includes pollen grains and endospores of organisms such as bacteria) may for instance be derived from a plant, or from a fungus, alga or bacterium or another micro-organism.

Suitable organisms from which such spores may be obtained include the following, with the approximate diameters of their spores, as published in the literature. Figures in brackets indicate the diameters measured by the inventors, where these differ from published values (e.g., P. D. Moore, J. A. Webb and M. E. Collinson, Pollen Analysis 2nd Edition, Blackwell Scientific Publications, Oxford, 1991).

| | | |
|---|---|---|
| Bacillus subtilis | 1.2 | μm |
| Myosotis (“forget-me-not”) | 2.4 (5) | μm |
| Aspergillus niger | 4 | μm |
| Penicillium | 3 (5) | μm |
| Cantharellus minor | 4 (6) | μm |
| Saccharomyces cerevisiae | 6 | μm |
| Ganomerma | 5 (6.5) | μm |
| Agrocybe | 10 (14) | μm |
| Urtica dioica | 10 (12) | μm |
| Periconia | 16 (18) | μm |
| Epicoccum | 20 | μm |
| Ryegrass “Lolium perenne” | 21 | μm |
| Timothy grass | 22 | μm |
| Rye | 22 | μm |
| Lycopodium clavatum L. | 34 | μm |
| “Lycopodium powder” | 40 | μm |
| Maize “Zea mays” | 80 | μm |
| Hemp “Cannabis sativa” | 24 | μm |
| Rape hemp | 25 | μm |
| Wheat | 23 | μm |
| Abies | 125 | μm |
| Cucurbitapapo | 200 | μm |
| Cuburbita | 250 | μm |

Of these, Lycopodium clavatum L., lycopodium powder, pine, ryegrass, rye, sunflower, Timothy grass, Ambrosia trifida L., Ambrosia artemisiifolia L., hemp, rape, wheat and maize spores may be preferred. Other spores from which dispersible exine shells may be extracted are disclosed in the publications referred to at page 8 of WO-2005/000280.

The dispersible exine shell may have a diameter (which may be determined by scanning electron microscopy or laser particle size analysis) of about 1 μm or greater, or of about 3 or 5 or 8 or 10 or 12 or about 15 μm or greater. It may have a diameter of up to about 300 μm, or of up to about 250 or 200 or 150 or 100 or 80 or 50 or about 40 μm. For example, its diameter may be from 1 to 300 μm, or from 1 to 250 μm, or from 3 to 80 μm, or from 3 to 50 μm, or from 15 to 40 μm. Grass pollen-derived dispersible exine shells, as well as other dispersible exine shells of approximately 20 μm diameter, might also be expected to be suitable, as may dispersible exine shells having diameters of up to around 80 μm.

The dispersible exine shells may have a % N level by weight of about 10% or less, or of about 8 or 6 or 4 or 2, or 1 or 0.5 or about 0.1% or less of the original spore.

In order to produce dispersible exine shells, the natural surface coating of the spores (including such as waxes and proteins) and natural encapsulated materials inside the naturally occurring spores, namely the cytoplasm may be at least partially, and preferably totally removed in the one-pot process.

The cytoplasm of the spores is made up of lipid, carbohydrate, protein and nucleic acid components that may be attached to or contained within the spores. It is desirable to remove as much of the cytoplasm as possible, in an efficient, short and economical one-pot process to form a hollow exine shell, which may then be utilised as a microcapsule.

Several different chemical methods, using a variety of chemical reagents, have been described. These methods attempt to produce empty exine shells from spores, for example:

F. Zetsche, P. Kalt, J. Liechti and E. Ziegler, J. Prakt. Chem., 148, (1937), 267-286, Saad M. Alshehri, Hamad A. Al-Lohedana, EidaAl-Farraj, Norah Alhokbany, Anis Ahmad Chaudhary, Tansir Ahamad. International Journal of Pharmaceutics 504, 39-47 (2016), Shashwati Atwe, Harvinder S. Gill, Yunzhe Ma, Patent WO2014062566 (A1), describes the use of sequentially, hot acetone, alkali and 85% phosphoric acid over several days; E. Dominguez, J. A. Mercado, M. A. Quesada and A. Heredia, Grana, Supplement 1, (1993), 12-17.) describes treating spores with anhydrous HF in pyridine; N. M. Tarlyn, V. R. Franceschi, J. D. Everard and F. A. Loewus, Plant, science, 90, (1993), 219-224, K. E. Espelie, F. A. Loewus, R. J. Pugmire, W. R. Woolfenden, B. G. Baldi and P. H. Given, Phytochemistry, 28, (1989), 751-753, and F. A. Loewus, B. G. Baldi, V. R. Franceschi, L. D. Meinert and J. J. McCollum, Plant Physiol., 78, (1985), 652-654.) describe treating spores with 4-methylmorpholine-N-oxide monohydrate; G. Erdtman, Svensk Botanisk Tidskrift, 54, (1960), 561-564, describes using a 9:1 mixture of acetic anhydride and concentrated sulphuric acid on spores, which have had contaminants mechanically removed; M. Couderchet, J. Schmalfus and P. Boger, Pesticide Biochemistry and Physiology, 55, (1996), 189-199, S. Gubatz, M. Rittscher, A. Meuter, A. Nagler and R. Wiermann, Grana, Supplement 1, (1993), 12-17, K. Schulze Osthoff and R. Wiermann, J. Plant Physiol., 131, (1987), 5-15 and F. Ahlers, J. Lambert and R. Wiermann, Z. Naturforsch., 54c, (1999), 492-495) describe methods which utilise enzymes; Michael G. Potroz, Raghavendra C. Mundargi, Jurriaan J. Gillissen, Ee-Lin Tan, Sigalit Meker, Jae, H. Park, Haram Jung, Soohyun Park, Daeho Cho, Sa-Ik Bang Nam-Joon Cho Advanced Functional Materials 27 (2017), Raghavendra C. Mundargi, Michael G. Potroz, Jae Hyeon Park, Jeongeun Seo, Ee-Lin Tan, Jae Ho Lee & Nam-Joon Cho Sci Rep. 2016; 6: 19960, Arun Kumar Prabhakar, Hui Ying Lai, Michael G. Potroz, Michael K. Corliss, Jae HyeonPark, Raghavendra C. Mundargi, Daeho Cho, Sa-Ik, Bang, Nam-Joon Cho Journal of Industrial and Engineering Chemistry 53, 375-385 (2017) describes acidolysis in phosphoric acid at 70° C. over the range for 5-30 h. Gill, Harvinder Singh, Atwe, Shashwati U., Gonzalez-cruz, Pedro E. US 2018/0092852 A1 2018, Gonzalez-Cruz, P., Uddin, M. J., Atwe, S. U., Abidi, N. & Gill, H. S. ACS Biomaterials Science & Engineering 4, 2319-2329 (2018). describes using hot phosphoric acid before hot potassium hydroxide solution in contrast to earlier methods of base hydrolysis followed by phosphoric acid; Mujtaba, M., Sargin, I., Akyuz, L., Ceter, T. & Kaya, M. Materials Science & Engineering C-Materials for Biological Applications 77, 263-270 (2017), Mundargi, R. C. Raghavendra C. Mundargi, Michael G. Potroz, Jae Hyeon Park, Jeongeun Seo, Jae Ho Leeab and Nam-Joon Cho RSC Advances 6, 16533-16539 (2016), Park, J. H., Seo, J., Jackman, J. A. & Cho, N. J. Scientific Reports 6 (2016) uses hot acetone followed by treatments with 4 M or 6 M HCl) at 70° C. for 10-48 h and a further 24 h in the acid.

All of these methods produce darkened exines shells, from light brown to very dark brown in colour. Importantly, none of these methods produces exine shells that disperse in solution.

Accordingly, a first aspect of the present invention is a method for extracting dispersible exine shells of naturally occurring spores comprising the steps of:
(a) treating in a one-pot process naturally occurring spores, with preferably an aqueous nonacidic treatment at a pH greater than pH 7 under nonreflux conditions (<85° C.) without the addition of an organic solvent. Where the nonacidic treatment may be either a base or surfactant or both, with or without a catalyst, which can be added at the same time or sequentially to the same one pot;
(b) separating the dispersible exine shells from the mixture after treatment.

Therefore, the present invention has the advantage of producing a product that disperses in a solvent or a solution with further advantages of being energy efficient, avoiding transfer of the contents of one reaction vessel to another, employing inexpensive non-toxic reagents and using simple filtration equipment. The dispersible exine shells may be washed after step (b), for example to remove any waste produced, remaining after step (a). The dispersible exine shells may be washed with water and/or an alcohol (e.g. methanol, ethanol, or isopropanol). Additionally, or alternatively the method may further comprise an additional step (c) during which the dispersible exine shells may be subjected to a drying step. For example, the dispersible exine shells may be dried in either air or under vacuum or a combination of both. Alternatively, the shells may be dried by spray-drying or lyophilisation. In a particular example, the dispersible exine shells may be dried using a desiccant, for example phosphorous pentoxide, calcium chloride and/or silica gel.

In the one-pot method of the invention, the reagents may be applied by means of the following non-limiting examples:
(i) base alone; or
(ii) surfactant alone; or
(iii) base and surfactant at the same time; or
(iv) base and then addition of surfactant; or
(v) surfactant and then addition of base

In one embodiment, the base or surfactant composition used in the present invention is an aqueous base or surfactant composition. Suitable base compositions include such as alkali bicarbonate, alkali carbonate or alkali hydroxide, calcium carbonate and ammonium hydroxide. Suitable surfactant compositions include alkyl benzene sulfonates, alkyl sulfates, alkyl ether sulfates, alkylbenzene sulfonates, alpha-olefin sulfonates, alkyl poly(ethylene glycol) ethers, sodium dodecyl sulfate ether sulfates, alcohol ethoxylates, carboxylate salts (soaps), such as sodium stearate, sodium lauroyl sarcosinate and carboxylate-based fluorosurfactants such as perfluorononanoate, perfluorooctanoate, C12-15 pareth-5, C12-15 pareth-7, fatty alcohol ether surfates, fatty alcohol ether sulfonates, fatty alcohol sulfonates, nonylphenyl poly(ethylene glycol) ethers, soap, sodium dodecylbenzenesulfonate, sodium acrylic acid/MA co-polymer, stearic acid, sodium polyarylsulfonate, sodium dodecyl sulfate, and sodium lauryl ether sulfate.

In an embodiment the base or surfactant composition may include a catalyst including but not limited to calcium bromide, calcium hydroxide, calcium molybdate or tungstate, ferric chloride, magnesium sulfate, potassium bromide, potassium hydroxide, potassium molybdate or tungstate, sodium bromide, sodium hydroxide, sodium molybdate or tungstate, manganese 1,4,7-triazacyclononane or iron 3,7-diazabicyclo[3.3.1]nonan-9-one.

Following one-pot treatment with the base or surfactant or both, with or without a catalyst, the dispersible exine shells may be used without further treatment but are preferably washed and then dried.

The dispersible exine shells of the present invention may be formulated with conventional additives appropriate for the application envisaged. In particular, there may advantageously be employed brightening agents such as optical brightening agents, fluorescent brightening agents and fluorescent whitening agents typically used to enhance the appearance of fabric and paper. Such agents are intended to cause a “whitening” effect by making materials look less yellow and increasing the amount of light reflected to the eye. Suitable brightening agents will be well known to those skilled in the art. Examples include stilbenes and fluorescent dyes such as umbelliferone, which adsorb energy in the UV portion of the spectrum and re-emit it in the blue portion of the visible spectrum. More specifically, there may be employed triazine-stilbenes (di-, tetra- or hexa-sulphonated), biphenyl-stilbenes, biphenylcoumarins, imidazolines, diazoles, triazoles and benzoxazolines.

The scope of the present invention is not limited by any particular usage of the dispersible exine shells to protect and/or deliver active substances. Examples of suitable uses are disclosed in WO-2005/000280, WO-2007/012856, WO-2007/012857 and PCT Application No. PCT/GB2008/004150, the contents of which are incorporated herein by reference.

In particular, the dispersible exine shells may be used as delivery vehicles. There are inherent advantages to the use of naturally occurring dispersible exine shells as delivery vehicles, as described in WO-2005/000280 (for example at pages 3 and 4 and in the paragraph spanning pages 5 and 6) and WO-2007/012857 (see pages 4 to 5). Because of its inherent non-toxicity, for instance, a spore-derived dispersible exine shell can be particularly suitable for use as a delivery vehicle in the context of formulations, which are likely to come into contact with, or be ingested by, the human or animal body or plants. The proteinaceous materials, which can otherwise cause allergic reactions to spores are preferably removed during the processes used to isolate the exine component.

Furthermore, the use of dispersible exine shells in the delivery of an active substance as herein described, may be advantageous because, inter alia, the dispersible exine shells may protect an active substance from the acidic stomach of an animal; the dispersible exine shells may aid in delivery of an active substance to the gut walls of an animal; the dispersible exine shells may slow the release of the active substance in the gut of the animal and enhance bioavailability; etc.

Naturally occurring dispersible exine shells make ideal candidates for the systemic delivery of active substances such as agrochemicals, pharmaceuticals, veterinary or nutraceuticals. They can also be of value for the topical delivery of active substances, since they have been found capable of releasing an encapsulated active on application of only moderate pressure, for example gentle rubbing, as described in WO-2007/012857, in particular at page 3.

The dispersible exine shells prepared from any given organism also tend to be very uniform in size, shape and surface properties, unlike typical synthetic encapsulating entities. There is, however, significant variation in spore size and shape, and in the nature of the pores in the dispersible exine shells, between different species, allowing a formulation according to the invention to be tailored dependent on the nature and desired concentration of the active substance, the site and manner of intended application, the desired active substance release rate, the likely storage conditions prior to use and the like.

It can also be possible to encapsulate relatively high quantities of an active substance within even a small dispersible exine shell. The combination of high active loadings, small encapsulant size and adequate protective encapsulation is something that can be difficult to achieve using other known encapsulation techniques, and yet can be extremely useful in the context of preparing for example, agrochemical, pharmaceutical, veterinary or dietetic preparations, foods or beverages.

As described above, a dispersible exine shell is generally inert and non-toxic. Sporopollenin, for example, which is a component of most spore exine shells, is one of the most resistant naturally occurring organic materials known to man. It can survive very harsh conditions of pressure, temperature and pH as well as being insoluble in most inorganic and organic solvents (see G. Shaw, “The Chemistry of Sporopollenin” in Sporopollenin, J. Brooks, M. Muir, P. Van Gijzel and G. Shaw (Eds), Academic Press, London and New York, 1971, 305-348). However, sporopollenin may be decomposed by strong oxidising agents.

The ready, and often inexpensive, availability of spores, together with their natural origin, also make them highly suitable for use as active substance protection and/or delivery vehicles.

Thus, the dispersible exine shell of the invention offers an opportunity for a novel formulation comprising of dispersible exine shells capable of the protection and/or delivery of active substances. Such active substances includes but is not limited to pharmaceutically or veterinary active substances (which includes drugs, vaccines and antibodies), nutraceutically active substances, dietetic active substances (which includes nutraceutically active substances), probiotics, living and dead cells (which includes human, animal, algal, bacterial, fungal or plant cells), virus genetic material, foods and food ingredients, food supplements, agrochemicals, herbicides, pesticides and pest control agents, plant treatment agents such as growth regulators, antimicrobially active substances, fertility and reproduction regulators (excluding immunocontraceptive vaccines), cosmetics, toiletries, disinfectants, detergents and other cleaning agents, construction materials, adhesives, diagnostic agents, dyes and inks, fuels, explosives, propellants and photographic materials. In general, the present invention may be used to protect an active substance that is chemically or physically bound to, or encapsulated within the cavity or wall of a dispersible exine shell of a naturally occurring spore, whether living, non-living, monomeric, oligomeric or polymeric and whether organic, inorganic or organometallic.

The active substance may itself be a naturally occurring substance or derived from a natural source, in particular a plant source.

The dispersible exine shell of the present invention may be particularly suitable as a protection and/or delivery and/or removal vehicle in a cosmetic product. A cosmetic product may for example be selected from makeup products (for example foundations, powders, blushers, eye shadows, eye and lip liners, lipsticks, other skin colourings and skin paints), skin care products (for example cleansers, moisturisers, emollients, skin tonics and fresheners, exfoliating agents and rough skin removers), fragrances, perfume products, essential oils, sunscreens and other UV protective agents, self-tanning agents, after-sun agents, anti-ageing agents and anti-wrinkle agents, skin lightening agents, topical insect repellents, hair removing agents, hair restoring agents and nail care products such as nail polishes or polish removers. A perfume product may comprise more than one fragrance. By way of example only, the dispersible exine shell of the present invention used as a delivery vehicle in a cosmetic product, may be a loaded dispersible exine shell for the delivery of a cosmetic material. The dispersible exine shell of the present invention used as a removal vehicle in a cosmetic product, may be an empty dispersible exine shell for the removal of bodily fluids, e.g. sweat. It will be understood by the person skilled in the art that a cosmetic product may comprise a combination of loaded dispersible exine shells and empty dispersible exine shells; for the delivery of a cosmetic material and separate, simultaneous or sequential removal of bodily fluids.

The dispersible exine shell of the invention may be used as a protection and/or delivery and/or removal vehicle in a toiletry product, which may be selected from soaps; detergents and other surfactants; deodorants and anti-perspirants; lubricants; fragrances; perfume products; dusting powders and talcum powders; hair care products such as shampoos, conditioners and hair dyes; and oral and dental care products such as toothpastes, mouthwashes, breath fresheners and coatings for dental flosses and tapes.

The dispersible exine shell of the invention may be used as a protection and/or delivery and/or removal vehicle in a household product. Such a product may, for example, be selected from disinfectants and other antimicrobial agents, fragrances, perfume products, air fresheners, insect and other pest repellents, pest control agents and fertility and reproduction regulators (excluding immunocontraceptive vaccines), pesticides, plant fertilisers, plant growth regulators, laundry products (e.g. washing and conditioning agents), fabric treatment agents (including dyes), cleaning agents, UV protective agents, paints and varnishes.

The dispersible exine shell of the invention may be used as a protection and/or delivery and/or removal vehicle in an agrochemical product. Such a product may, for example, be selected from herbicides, fungicides, insect and other pest repellents, pesticides and pest control agents, fertility and reproduction regulators (excluding immunocontraceptive vaccines) and plant and soil treatment agents such as fertilisers, liming and acidifying agents (which are designed to change the pH), soil conditioners and growth regulators and chemicals used in the raising of livestock such as antibiotics and hormones.

The dispersible exine shell of the invention may be used as a protection and/or delivery vehicle for incorporation of an active substance into an animal bait.

The dispersible exine shell of the invention may be used as a protection and/or delivery and/or removal vehicle for genetic material.

The dispersible exine shell of the invention may be used as a protection and/or delivery vehicle for an active substance that is an agrochemical, a pharmaceutical (which includes drugs, vaccines and antibodies) or dietetic (which includes nutraceutical) active substance, which includes substances for veterinary use. The active substance may be a pharmaceutically active substance, which is suitable for topical delivery, for example selected from substances for use in treating skin or skin structure conditions (for example acne, psoriasis or eczema), wound or burn healing agents, anti-inflammatory agents, anti-irritants, antimicrobial agents (which can include antifungal and antibacterial agents), vitamins, vasodilators, topically effective antibiotics, antiseptics and agents providing skin protection against solar radiation. The active substance may be suitable and/or intended and/or adapted for oral delivery. It may therefore be suitable and/or intended and/or adapted for ingestion, by either humans or animals but in particular by humans. The active substance may be suitable and/or adapted and/or intended for anal, buccal, intramuscular, intraperitoneal, intravenous oral, pulmonary, nasal, inhalation, subcutaneous, transdermal, transmucosal, vaginal, or any other suitable form of delivery.

A pharmaceutically or nutraceutically active substance may be suitable and/or intended and/or adapted for either therapeutic (e.g., healing, curing, remedial, medicinal, restorative, health-giving, tonic, sanative, reparative, corrective, ameliorative drugs) or prophylactic use such as a vaccine.

The dispersible exine shell of the invention may be used as a protection and/or delivery vehicle for an active substance that is a diagnostic agent, in particular one intended for oral ingestion. For instance, the active substance may be a biological marker, a radioactive tracer, or a magnetic tracer for use in magnetic resonance imaging. In such cases, a protective additive may help to ensure that the co-encapsulated active substance reaches its intended delivery site.

The dispersible exine shell of the invention may be used as a protection and/or delivery vehicle for incorporation of a substance into a food or beverage product, which terms include food and beverage ingredients. Food and beverage ingredients may include, for example, dietary supplements (such as vitamins and minerals, folic acid, omega-3 oils, fibre or so-called “probiotics” or “prebiotics”), flavourings, fragrances, essential oils, colourings, preservatives, stabilisers, emulsifiers and agents for altering the texture or consistency of a food product.

The dispersible exine shell of the invention may be used as a protection and/or delivery vehicle for incorporation of a substance into a confectionery or chewing gum product.

The dispersible exine shell of the invention may be used as a protection and/or delivery vehicle for incorporation of a substance that is a hydrophilic and/or hydrolysable and/or acid-labile substance, or any other substance, which is at least partially degraded or otherwise altered in the presence of gastric fluid. It may for example be a proteinaceous material, which term includes (i) proteins, peptides, oligopeptides and polypeptides, or (ii) nucleic acids, oligonucleotides, nucleosides and nucleotides. It may be a carbohydrate, which term includes mono-, di-, oligo- and poly-saccharides as well as more complex carbohydrates such as gangliosides and cerebrosides; a lipid (e.g. a phospholipid, steroid, terpene or carotenoid); a nucleoside, nucleotide or nucleic acid; a vitamin or co-vitamin such as ascorbic acid or vitamin B 12; an essential fatty acid such as an omega-3 oil; an essential mineral or mineral-containing substance such as one containing iron, calcium, magnesium or zinc; a glyconutrient; a phytonutrient; another nutritional agent such as folic acid; or a microorganism such as a bacterium.

Particular examples include peptides, oligopeptides and proteins (e.g., hormones such as insulin and growth hormones such as somatropin); antibodies; antibiotics cells, stem cells, vaccines; enzymes (e.g. lactase and alkaline phosphatase); probiotics (e.g. Lactococcus lactis, a Gram-positive bacterium); and prebiotics (e.g. carbohydrates such as lactulose, lactitol oligofructose, inulin and galacto-oligosaccharides, tagatose, isomalto-oligosaccharides, polydextrose and maltodextrin). Particular examples of nucleic acids and analogues include antisense oligonucleotides (e.g. eteplirsen) with either RNA or DNA sequences or masked nucleotides (e.g. Sofosbuvir) or phosphonate nucleotides (cyclic cidofovir) used in such as antiviral chemotherapy or vaccines (excluding immunocontraceptive vaccines).

The dispersible exine shell of the invention may be used as a protection and/or delivery and/or removal vehicle for an active substance, which is a volatile substance, in particular an essence, e.g. a flavour or fragrance. The present invention can be particularly suitable for formulations containing such substances as the dispersible exine shells can help to inhibit release of any volatile components prior to use.

An active substance may be chemically or physically bound to, or more particularly encapsulated within, the cavity or shell of dispersible exine shell according to the invention. Chemical and physical binding may be achieved for example in the ways described in WO-2005/000280, WO-2007/012856 and WO-2007/012857; they may involve chemical modification of the dispersible exine shell or at least of its outer surface.

A substance may be encapsulated within a dispersible exine shell using known techniques, again suitably as described in WO-2005/000280. Conveniently, prepared dispersible exine shells may be immersed in a solution or suspension of the relevant substance, which is then allowed to impregnate the shells, suitably followed by a drying step to remove at least some of the residual solvent(s). Where the substance to be encapsulated is a liquid, such as an oil, the prepared dispersible exine shells may simply be immersed in the liquid, which they will then absorb. The rate of absorption can be increased with the aid of a vacuum.

The dispersible exine shells are suitably immersed in an excess of the substance to be encapsulated within them. One or more penetration enhancing agents may be used, again as described in WO-2005/000280, to aid impregnation of the dispersible exine shell by the relevant substance. A reduced or increased pressure (with respect to atmospheric pressure) may instead or in addition be used to facilitate impregnation; for example, a mixture of dispersible exine shells and an active substance may be placed under vacuum in order to increase the rate of absorption of the active by the dispersible exine shells.

A substance may be generated in situ within a dispersible exine shell, for instance from a suitable precursor substance already associated with the dispersible exine shell. For example, a precursor substance may be chemically or physically bound to, or encapsulated within, the cavity or the shell of a dispersible exine shell, which is then contacted with a reactant substance that reacts with the precursor to generate the desired active substance or additive.

The dispersible exine shell may be loaded with, or otherwise associated with, any suitable quantity of an active substance, depending on its intended use. A formulation according to the invention may, for example, contain an active substance and dispersible exine shells at a weight ratio of from 0.0001:1 to 15:1, such as from 0.0001:1 to 10:1, or from 0.0001:1 to 5:1, or from 0.001:1 to 5:1 or 0.01:1 to 5:1 or from 0.1:1 to 5:1 or 0.5:1 to 5:1. Larger dispersible exine shells may be needed in order to achieve higher active substance loadings.

The dispersible exine shell may be coated with a barrier layer, for example for further protection of an associated active substance, to prevent its release until a desired time or location is reached, or for taste masking purposes. Such coatings may be as described in WO-2005/000280, WO-2007/012856 or WO-2007/012857. The coating may be attached by ionic, covalent, hydrogen bonding or van de Waals forces or hydrophobic-hydrophilic interactions.

Where the dispersible exine shell of the invention is used as an antioxidant, it can simply be contacted with a substance or composition to be protected. Instead or in addition, the substance or composition can be chemically or physically bound to, or encapsulated within, the cavity or in the shell of a dispersible exine shell. Suitable such methods are described in WO-2007/012856.

A formulation according to the second aspect of the invention may contain more than one active substance. For example, two or more such active substances may be chemically or physically bound to or co-encapsulated in the same dispersible exine shell or within the shell wall. Instead or in addition, a formulation prepared according to the invention may comprise two or more populations of active substance-containing dispersible exine shells, each chemically or physically bound to, or encapsulating, a different active substance. A formulation prepared according to the invention may comprise two or more populations of active substances where one or more of the active substances are not chemically or physically bound to, or encapsulated in a dispersible exine shell. Thus, the active substance is absent and the dispersible exine shell is empty.

It is within the scope of the present invention for a formulation to contain two or more active substances, alternatively the formulation may comprise one active substance in combination with an additional active substance.

According to one aspect of the invention there is provided a formulation as herein described wherein the one or more active substances are encapsulated within the cavity of a dispersible exine shell or within the shell wall.

According to one aspect of the invention there is provided a formulation as herein described wherein the one or more active substances are chemically or physically bound to the dispersible exine shell and/or encapsulated within the dispersible exine shell or within the shell wall.

According to another aspect of the invention there is provided a formulation as herein described comprising two or more active substances.

According to this aspect of the invention in the formulation two or more active substances may be chemically or physically bound to, or encapsulated within the same dispersible exine shell or within the shell wall.

According to another aspect of the invention in the formulation a first active substance is chemically or physically bound to, or encapsulated within a first dispersible exine shell or within the shell wall; and a second active substance is chemically or physically bound to, or encapsulated within a second dispersible exine shell or within the shell wall.

According to another aspect of the invention in the formulation a first and a second active substance is absent and the dispersible exine shells are empty.

According to another aspect of the invention in the formulation a first active substance is absent from a first dispersible exine shell or within the shell wall and the dispersible exine shell is empty; and a second active substance is chemically or physically bound to, or encapsulated within a second dispersible exine shell or within the shell wall.

When the formulation of the invention comprises dispersible exine shells wherein an active substance is not chemically or physically bound to or encapsulated within the dispersible exine shells or dispersible exine shell wall, such dispersible exine shells are considered to be empty dispersible exine shells that can be available for removal, e.g. remediation or sequestration as herein defined, for example for the removal of animal or human bodily fluids (including such as blood, faeces, saliva, sweat, tears or urine).

Thus, for example, a cosmetic formulation prepared according to the invention might contain both a sunscreen and an insect repellent, or a sunscreen and a moisturiser, or a foundation or other skin colouring agent and a sunscreen. Two or more active substances may therefore benefit from the antioxidant protection afforded by the dispersible exine shells. This can also enable two or more active substances to be kept separate prior to use, and then released together in situ at the intended point of use, which may, for example, be of value if the two or more active substances are incompatible with one another or would interact in an undesirable manner. Alternatively, the antioxidant protection afforded by the dispersible exine shells can enable two or more active substances to be kept apart before reaching the site of action and then released where they interact in a desirable manner, e.g., certain adhesives, dyes, paints, etc.

A formulation according to the second aspect of the invention may contain one or more additional agents for instance selected from fluid vehicles, excipients, adjuvants, diluents, carriers, stabilisers, surfactants, penetration enhancers or other agents for targeting delivery of the dispersible exine shell and/or an associated active substance to the intended site of administration or action.

The formulation may take the form of a lotion, cream, ointment, paste, gel, foam, powder, suspension or any other physical form known for topical administration, including for instance a formulation which is, or may be, applied to a carrier such as a sponge, swab, brush, tissue, skin patch, dressing or dental fibre or tape to facilitate its topical administration. It may take the form of a viscous or semi-viscous fluid, or of a less viscous fluid such as might be used in sprays (for example nasal sprays or body sprays), drops (e.g., eye or eardrops), aerosols or mouthwashes.

The formulation may alternatively take the form of a powder, for example, when the active substance is a makeup product such as a blusher, eye shadow or foundation colour, or when it is intended for use in a dusting powder. Exine shells can be extremely efficient at absorbing liquids, in particular lipids, to result in an effectively dry product with all of the liquid encapsulated within the shells, as demonstrated in Example 11 of WO-2007/012856. Other active substances, for example food or beverage supplements or ingredients, or agrochemicals, or pharmaceutically, veterinary or nutraceutically active substances, may also be formulated as powders.

For oral delivery, the formulation may for example take the form of a tablet, gel, paste, capsule, lozenge, solution or suspension, or of a food (including an animal feed) or beverage. Other suitable pharmaceutical and dietetic dosage forms are those disclosed in WO-2005/000280, for instance at pages 3 and 6 to 9.

In accordance with the invention, tablets can be made by tightly compressing an amount of dispersible exine shells with other ingredients. For example, a tablet may also include inactive fillers, binders, diluents, lubricants, disintegrants, colouring agents and flavouring agents. An inert filler may be used to obtain the precise size and shape of the tablet desired. The filler is usually selected from a group of compounds that are inert with respect to the active compound that will be encapsulated. The formation of a tablet may be by any method that is known in the art for forming tablets. The most common method is the compression method. In the compression method, the components of a tablet are mixed, either wet or dry, and then (can be followed by drying in the case of wet mixing) an amount of the composition is applied to a mould. A die then compresses the mixture in the mould, forming a tablet. If the process is a wet process, the composition or tablet must be dried. A tablet can also be coated as described above by those methods known in the art for coating tablets. The most common method of making a tablet involves mixing the ingredients of the tablet and compressing the mixture in a mould to give it the desired shape and hardness. The mixture of ingredients is usually mechanically compressed by a machine. The compressed mixture may be either wet or dry. However, in a method where the mixture is wet, the mixture or tablet must be dried. Lubricants can be added to the composition that is to be formed into a tablet to help reduce the frictional wear of the die and its associated parts. Binders may also be added to help promote the adhesion of the different ingredients of the mixture.

Some of the commonly used diluents include, but are not limited to, spray dried dextrose, lactose, calcium triphosphate, sodium chloride and microcrystalline cellulose. Some commonly used binders include, but are not limited to, acacia, ethyl cellulose, gelatine, glucose syrups, starch mucilage, polyvinyl pyrrolidone, sodium alginate and sucrose syrups. Some commonly used lubricants include, but are not limited to, magnesium stearate, stearic acid, talc, vegetable oils, colloidal silica and polyethylene glycol. Some commonly used disintegrants include, but are not limited to, starch, alginic acid, microcrystalline cellulose, crospovidone and sodium lauryl sulfate. Some commonly used inert fillers include, but are not limited to, silica, Avicel™ (microcrystalline cellulose), lactose, starch and mannitol.

Disintegrants help the tablet to disintegrate in vivo and thus help to deliver the therapeutically active compound contained in the tablet. It is also sometimes desirable to coat the tablet. The coating may, for example, be a thin film of light-sensitive material to prevent decomposition of the tablet. Alternatively, the film may have other purposes such as to mask a tablet's unpleasant taste or to delay the disintegration or dissolution of a tablet. A delay may be important for some therapeutically active compounds that irritate the stomach, and it may be desirable to delay the dissolution of the tablet containing such a compound until the tablet reaches the intestine.

If desired, the dispersible exine shell may contain one or more protective additives that are co-encapsulated with an active substance, as described in PCT Application No. PCT/GB2008/004150. The active substance and the additive may be encapsulated within the dispersible exine shell either simultaneously or sequentially. In the former case, the active substance and additive may be mixed together, if necessary, in an appropriate solvent system, and the mixture then encapsulated within the dispersible exine shell, for instance, using the immersion technique described above. In the latter case, the dispersible exine shell may be impregnated firstly with the active substance or a solution or suspension thereof and secondly with the protective additive or a solution or suspension thereof, if necessary, with a drying step between the two impregnation steps.

It may be preferred for the active substance to be encapsulated before the additive, as this may serve to increase the protective effect of the additive. It is believed that, in such cases, the additive may form an, at least partial protective layer around the outside of an active substance “core” and that, in some cases, the additive may, at least partially coat the inside of the dispersible exine shell, thus blocking at least some of its pores.

In the foregoing, a “suspension” of an active substance or additive may be a dispersion, emulsion or any other multi-phase system.

The additive may be a substance that is either solid or semi-solid under the normal storage conditions for the formulation (typically at room temperature). It may melt at a higher temperature (for instance, body temperature) at which the active substance is intended to be released from the formulation—examples of materials that behave in this way include cocoa butter and various fatty acids. The additive may be a material, which is capable of masking, at least partially, the flavour and/or aroma of a co-encapsulated active substance.

Particularly suitable protective additives include (a) acrylic-based polymers such as the poly(alkyl)acrylates or poly(alkyl cyanoacrylates) or poly(meth)acrylates, in particular the polymers available under the trade name Eudragit® (Evonik Industries); (b) cellulosic materials, in particular cellulose-based polymers such as the cellulose acetate phthalates; (c) lipids including isoprenoid-based materials (for example materials based on terpenes and steroids) and fatty acid-based materials including fatty acids themselves and amides and esters of fatty acids (including mono-, di- and tri-glycerides and phospholipids); (d) materials having a lipid component, for example a lipid side chain, in particular fatty acids, e.g. lipoproteins, glycoproteins and shellac; (e) polysaccharides such as cellulose, chitin, chitosan, starch, heparin and gum Arabic; and (f) other synthetic polymers including polyoxyalkylene-based surfactants, polymethylsiloxane, polyvinyl pyrrolidone, polyvinyl alcohol, ethylene/vinyl acetate copolymer, polyesters, polyurethanes, polycarbonates, polystyrene, polyols, polythiols, polyamines, polyethylene, polypropylene, poly(lactic acid), poly(lactic co-glycolide acid), polyglutamic acid, soya bean protein, hydrolysates and poly FA-SA (poly fumaric acid-sebacic acid).

It will be appreciated that certain protective additives may fall within two or more of the above general classes or may contain a mixture of components, which themselves fall into different categories. Thus, for example cellulose itself is a polysaccharide (type e) but gives rise to the class (b) of cellulosic materials.

In WO-2007/012856 it is disclosed that exine shells can themselves act as antioxidants and provide protection for example against UV-induced oxidation. This effect is believed to be additional to the physical barrier provided by the exine shell limiting the ingress of air. For many applications, it is highly desirable to combine the ability to provide antioxidant protection with a dispersible exine shell. The invention thus provides the use of such a dispersible exine shell as an antioxidant protection, and/or as a delivery vehicle for an active substance, which is sensitive to oxidation.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally, the invention extends to any novel one, or any novel combination, of the features disclosed in this specification (including any accompanying claims and drawings). Thus, features, integers, characteristics, compounds, chemical moieties or groups described in conjunction with a particular aspect, embodiment or example of the invention are to be understood to be applicable to any other aspect, embodiment or example described herein unless incompatible therewith.

Moreover, unless stated otherwise, any feature disclosed herein may be replaced by an alternative feature serving the same or a similar purpose.

The present invention will now be described by means of the following non-limiting examples (where Examples 1-5 are one-pot extraction methods of the present invention and Comparisons 1 to 5 are two-pot extraction methods:

### EXAMPLES

Dispersibility of the exine shell products of the Examples and Comparisons was measured.

A 100 mL measuring cylinder was filled up to 50 mL with water (18° C.). Exine shell products from Comparisons 1 to 5 and Examples 1 to 5 were separately measured into vials with an internal diameter of 1.6 cm and filled up to a height of 2 cm such that the total volume of each exine shell product was 4.02 cm³. The mass of each exine shell powder was then recorded, and the samples were poured quickly and gently on to the top of the water layer of the measuring cylinder using a long funnel. The time taken for each exine shell product to go below the meniscus without agitation was then measured and the results are shown in Table 1.

Comparison 1

Raw Lycopodium clavatum L. spores (600 g) were stirred for 1 h in 6M HCl aqueous solution (2.9 L) at 94° C. The product was filtered under vacuum (porosity grade 2) and washed with hot water (60-70° C.) (500 ml×2), 6% NaOH (w/v) aqueous solution (500 ml×2) (NB check the pH of filtrate was basic), hot water (60-70° C.) (500 ml×2) or until neutral pH of filtrate, methanol (1 L×2), acetone (1 L×2). The product was then dried under vacuum overnight before further drying in an oven at 50° C. until constant weight to yield 300 g (50%) (Thomasson, et al., 2020).

Comparison 2

Raw Lycopodium clavatum L. spores (200 g) were stirred for 4 h in acetone (800 mL) at 60° C. The defatted sporopollenin (DFS) was filtered (porosity grade 2) and air-dried under vacuum over phosphorous pentoxide. DFS (200 g) was then stirred for 6 h in 6% (w/v) KOH aqueous solution (900 ml) at 80° C. The product was filtered under vacuum (porosity grade 2) and washed with hot water (60-70° C.) (500 mL×2). This operation was repeated with fresh 6% (w/v KOH aqueous solution (900 mL). The product was filtered (porosity grade 2) and washed with hot water (60-70° C.) (500 ml×6) and was then stirred for 5 days in 85% H₃PO4 (900 mL) at 60° C. The product was filtered (porosity grade 2) and washed with hot water (60-70° C.) (500 mL×2), 2M NaOH (300 mL×2), hot water (60-70° C.) (500 mL×2) or until neutral pH of the filtrate, PBS (250 mL×2), hot water (60-70° C.) (500 mL×2), ethanol (250 mL×2). The product was stirred for 4 h in ethanol (900 ml) at 80° C. and filtered under vacuum (porosity grade 2) and washed with ethanol (250 mL×2) and acetone (250 mL×2). The product was sonicated for 30 min in acetone (500-700 ml) and filtered under vacuum overnight (over phosphorous pentoxide) before further drying in an oven at 50° C. until constant weight to yield 70 g (35%) (Bailey, et al., 2019).

Comparison 3

As described in Barrier 2008 raw Lycopodium clavatum L. spores (200 g) were stirred for 6 h in 6% NaOH (w/v) aqueous solution (800 ml) at reflux. The product filtered under vacuum (porosity grade 2) and stirred for a further 6 h in fresh 6% NaOH (w/v) aqueous solution at reflux. The product was filtered under vacuum (porosity grade 3) and washed with hot water (60-70° C.) (300 ml×3) and hot ethanol (ca 60° C.) (300 ml×3), and then stirred for 2 h in ethanol at reflux. The product was filtered under vacuum (porosity grade 2) and dried in an oven at 60° C. for 12 h to yield 80 g (40%).

Comparison 4

Helianthus annuus L. bee pollen pellets (1.2 Kg) were stirred for 2 h in hot water (3.5 L) at 80° C. The defatted sporopollenin (DFS) was filtered under vacuum (porosity grade 2) and washed with hot water (60-70° C.) (500 mL×2), methanol (1 L×2) and acetone (1 L×2. The product was dried under vacuum (porosity grade 2) before further drying overnight in oven at 50° C. The product (50 g) was stirred for 1 h in 6 M HCl aqueous solution (230 mL) at 95° C. The product was filtered under vacuum (porosity grade 2) and washed with water (500 mL×2) or until neutral pH of the filtrate, and methanol (200 mL) and air-dried (12 h) before further drying in an oven at 50° C. until constant weight to yield 240 g (20%) (Mundargi et al., 2016).

Comparison 5

Defatted Pinus L. pollen (4 g) was stirred for 1 h in 85% ortho-phosphoric acid aqueous solution (30 mL) at 70° C. The product was filtered under vacuum (porosity grade 2) and washed with distilled water (150 mL×5), acetone (100 mL×1), 2 M HCl (100 mL×1), distilled water (100 mL×5), acetone (100 mL×1), and ethanol (100 mL×2) and then dried in an oven at 60° C. for 6 h to yield 1.6 g (40%) (Mundargi, et al., 2016; Lale & Gill, 2018; Prabhakar, et al., 2017).

The dispersibility of Comparisons 1 to 5 were measured as described above. The results are presented in Table 1.

### Example 1

Raw Helianthus annuus L. bee pollen (50 g) was stirred for 2 h in 2% w/w Fairy liquid (‘Original’ containing 15-30% anionic Surfactants, 5-15% non-ionic surfactants, benzisothiazolinone, phenoxyethanol, perfumes, limonene) aqueous solution (1 L) at 80° C. (pH>7). The product was filtered under vacuum (porosity grade 2) and washed with hot water (60-70° C.), before washing with cold water until the eluent appears colourless and of neutral pH. The product was then dried in an oven at 60° C. overnight to yield 10 g (20%).

### Example 2

Raw Lycopodium clavatum L. spores (50 g) were stirred for 16 h in 1% w/w sodium dodecyl sulfate aqueous solution (1 L) at 30° C. (pH>7). The product was filtered under vacuum (porosity grade 2) and washed with hot water (60-70° C.), before washing with cold water (ca 20° C.) until the eluent appeared colourless and of neutral pH. The product was then dried in an oven at 60° C. overnight to yield 35 g (70%).

### Example 3

Raw Pinus L. pollen (50 g were stirred for 16 h in 1% w/w sodium dodecyl sulfate aqueous solution (1 L) at 30° C. (pH>7). The product was filtered under vacuum (porosity grade 2) and washed with hot water (60-70° C.), before washing with cold water (ca 20° C.) until the eluent appeared colourless and of neutral pH. The product was then dried in an oven at 60° C. overnight to yield 35 g (70%).

### Example 4

Raw Lycopodium clavatum L. spores (500 g) were stirred for 16 hours in 6% (w/w) NaOH aqueous solution (2.8 L) at 71° C. The product was filtered under vacuum (porosity grade 2) and washed with hot water (60-70° C.) (500 mL×2), before washing with cold water (ca 20° C.) (500 mL×2) or until the eluent appears colourless and of neutral pH. The product was then dried in an oven at 60° C. overnight to yield 200 g (40%).

### Example 5

Raw Pinus L. pollen (5 g) was stirred for 10 min in 2% (w/w) NaOH aqueous solution (30 mL) at 80° C. The product was filtered under vacuum and washed with hot water (60-70° C.) (50 mL×2) before being washed with cold water (ca 20° C.) (50 mL×2) or until the eluent appeared colourless and of neutral pH. The product was then dried in an oven at 60° C. overnight to yield 3.2 g of (64%).

The dispersibility of Examples 1 to 5 were measured as described above. The results are presented in Table 1.

Table 1 below shows the results for all of Comparisons 1 to 5 and Examples 1 to 5.

TABLE 1
Dispersibility Measurements

| Sample | Time to disperse (seconds) * |
|---|---|
| Comparison 1 | ◯◯ |
| Comparison 2 | ◯◯ |
| Comparison 3 | ◯◯ |
| Comparison 4 | ◯◯ |
| Comparison 5 | ◯◯ |
| Example 1 | 36 |
| Example 2 | 12 |
| Example 3 | 24 |
| Example 4 | 19 |
| Example 5 | 43 |

* ◯◯—Signifies no observed dispersion after 1 hour of testing.

The data provided in Table 1 provide a quantitative test for dispersible exine shells. Within 60 seconds, the dispersible exine shell product (Example 1-5) falls below the meniscus without agitation whereas exine microcapsules extracted under reflex conditions (>85° C.) and/or multi-pot conditions did not disperse within this timeframe.

1. A formulation comprising one or more active substances together with a dispersible exine shell of a naturally occurring spore or pollen grain; and wherein the dispersible exine shell is dispersible in water at 18° C. wherein within 60 seconds, the dispersible exine shell falls below a meniscus without agitation; and wherein the active substance is not an immunocontraceptive.

2. A formulation according to claim 1 wherein the dispersible exine shell is extractable by a process comprising treating a spore or pollen grain with an aqueous nonacidic treatment at a pH greater than pH 7, under nonreflux conditions at <85° C., with or without a catalyst and wherein the method is a one-pot process.

3.-7. (canceled)

8. A formulation according to claim 1, wherein the one or more active substances are chemically or physically bound to the dispersible exine shell and/or encapsulated within the dispersible exine shell or within the shell wall.

9.-10. (canceled)

11. A formulation according to claim 1, comprising two or more active substances.

12. A formulation according to claim 11, wherein two or more active substances are chemically or physically bound to, or encapsulated within the same dispersible exine shell or within the shell wall of the same dispersible exine shell or a combination of these.

13. A formulation according to claim 11, wherein a first active substance is chemically or physically bound to, or encapsulated within a first dispersible exine shell or within the shell wall; and a second active substance is chemically or physically bound to, or encapsulated within a second dispersible exine shell or within the shell wall.

14. A formulation according to claim 11, wherein a first and a second active substance is absent and the dispersible exine shells are empty.

15. A formulation according to claim 11 wherein a first active substance is absent from a first dispersible exine shell or within the shell wall and the dispersible exine shell is empty; and a second active substance is chemically or physically bound to, or encapsulated within a second dispersible exine shell or within the shell wall.

16. A formulation according to claim 1, which is an agrochemical product, a beverage product, a cosmetic product, a household product, a toiletry product, a laundry product, a food product, a dietetic (which includes nutraceutical) product or a diagnostic, pharmaceutical, vaccine or veterinary product, a confectionery or chewing gum product.

17. (canceled)

18. A formulation according to claim 1 which is suitable and/or adapted and/or intended for anal, vaginal, oral, topical, intravenous, pulmonary, nasal, buccal, inhalation, sub-lingual, transdermal, transmucosal, subcutaneous, intramuscular, intraperitoneal or any other suitable form of delivery.

19.-20. (canceled)

21. A method of preparing a dispersible exine shell, the method involving isolating a dispersible exine shell from a naturally occurring spore or pollen grain by treating the spore with an aqueous nonacidic treatment at a pH greater than pH 7, under nonreflux conditions at <85° C. with or without a catalyst and wherein the method is a one-pot process.

22.-25. (canceled)

26. A method according to claim 21, wherein the dispersible exine shell is extractable without use of an organic solvent.

27. A method according to claim 21 wherein the aqueous nonacidic treatment of the spore or pollen grain, with or without a catalyst, is carried out in the same reaction vessel.

28. A method according to claim 21, wherein the aqueous nonacidic treatment of the spore or pollen grain, with or without a catalyst, can be added at different times in the same reaction vessel.

29. A method according to claim 21, wherein the aqueous nonacidic treatment, of the spore or pollen grain, with or without a catalyst, can be carried out at different temperatures in the same reaction vessel.

30. A method according to claim 21 wherein the extraction time is from 10 minutes to 24 hours.

31. A method according to claim 21 wherein the dispersible exine shell has been isolated from a naturally occurring spore or pollen grain under hydrolysis conditions.

32. A dispersible exine shell isolated from a naturally occurring spore or pollen grain wherein the exine shell is dispersible in water at 18° C. wherein within 60 seconds, the dispersible exine shell falls below a meniscus without agitation.

33. A dispersible exine shell according to claim 32 wherein the derivatisation of the dispersible exine shell comprises, hydrolysis, salt formation, protonation, deuteration, tritiation, esterification, amination, quarternisation, acetylation, sulfonation, sulfation, thiolation, alkylation, azidation, phosphorylation, nitration, metal chelation, halogenation, hydrogenation or chloromethylation or thiolation or any combination thereof.

34. A dispersible exine shell according to claim 32 wherein the dispersible exine shell is extractable by a process comprising treating a spore or pollen grain with an aqueous nonacidic treatment at a pH greater than pH 7, under nonreflux conditions at <85° C., with or without a catalyst and wherein the method is a one-pot process.

35.-37. (canceled)

38. A dispersible exine shell according to claim 32, wherein the dispersible exine shell is extractable without use of an organic solvent.

39. A dispersible exine shell according to claim 32, for use in a method of surgery, therapy, prevention or diagnosis practised on a living plant, human or animal body.

40. Use of a dispersible exine shell according to claim 32, as a protection, and/or delivery and/or removal vehicle for one or more active substances.

41. Use of a dispersible exine shell according to claim 40 as an antioxidant for an active substance.

42. Use of a dispersible exine shell according to claim 40, in the manufacture of a medicament for the protection and/or delivery of an agrochemical, pharmaceutically or veterinary active substance or a diagnostic agent to a human or animal patient.

43. Use of a dispersible exine shell according to claim 40 wherein a protective additive is, together with the active substance, chemically or physically bound to the dispersible exine shell and/or encapsulated within the dispersible exine shell or within the shell wall.

44.-45. (canceled)

46. Use of a dispersible exine shell as claimed in claim 40 wherein the outside of the dispersible exine shell is further coated with a material to aid retention of the active sub stance.

47. Use of a dispersible exine shell according to claim 40 in the removal of an active substance or animal bodily fluids or human bodily fluids.

48. Use of a dispersible exine shell according to claim 47 in the removal of an active substance from solutions and emulsions.

49. (canceled)

50. Use of a dispersible exine shell according to claim 40 in the manufacture of a formulation for the protection and/or delivery of an active substance to a living organism and/or delivery of an active substance to a non-living material.

51.-52. (canceled)

53. A method for protecting an active substance from oxidation and/or for increasing the stability of the active substance or of a composition containing it, the method comprising formulating an active substance with a dispersible exine shell according to claim 32.

54. A method of increasing the oxidative stability of an active substance, which comprises adding a dispersible exine shell as claimed in claim 32 to the active substance.

55. (canceled)
